(19) United States
(12) Patent Application Publication
(10) Pub. No.: US 2020/0046779 A1
(43) Pub. Date: Feb. 13, 2020

(54) ANTI-INFLAMMATORY EXOSOMES FROM INFLAMED CELLS OR TISSUES

(71) Applicants: Kayvan NIAZI, Culver City, CA (US); VBC HOLDINGS LLC, Culver City, CA (US)

(72) Inventors: Kayvan NIAZI, Encino, CA (US); Francesco CURCIO, Pagnacco (UD) (IT)

(73) Assignee: VBC HOLDINGS LLC, Culver City, CA (US)

(21) Appl. No.: 16/341,799

(22) PCT Filed: Oct. 12, 2017
(86) PCT No.: PCT/US2017/056358

§ 371 Date: Apr. 12, 2019

Related U.S. Application Data
(60) Provisional application No. 62/407,842, filed on Oct. 13, 2016.

(51) Int. Cl. A61K 35/38; C12N 5/0786

(52) U.S. Cl. CPC A61K 35/38; C12N 5/0679; C12N 5/0645

(57) ABSTRACT

The invention provides anti-inflammatory exosomes that, when administered locally or systemically to a subject diagnosed with an inflammatory disease or disorder, will downregulate the inflammatory process in the subject.

### TECHNICAL FIELD

The present disclosure relates generally to anti-inflammatory exosomes, methods of obtaining or producing anti-inflammatory exosomes, and to methods of treating a disease or disorder exhibiting or caused by an inflammatory process, by administering anti-inflammatory exosomes to a subject needing such treatment.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Inflammation is a normal response of the immune system to a wide variety of injuries, infection and/or other insults to living tissue. Generally, inflammation results from an acute injury or disease process, and the signs of inflammation, e.g., pain, heat, redness, swelling, and loss of function, are of limited scope and duration. The inflammatory reaction is mediated by a complex interplay of a variety of immune cells and chemical mediators, such as bradykinin and histamine, as well as various cytokines. Unfortunately, some types of injury and/or disease processes, particularly those that are long lasting and chronic in nature, can provoke a corresponding long lasting inflammatory process in living tissue that will cause further damage to the affected and surrounding tissues, organs, or the entire organism.

Chronic inflammation is associated with, or found to cause a wide range of disease processes, both localized and systemic, in nearly every organ system. For example, metabolic syndrome (also referred to as, “metabolic X syndrome”) is a chronic condition that can occur in mammals, including humans, that exhibit chronic above normal central fat deposits and that receive insufficient exercise. Metabolic syndrome is a systemic inflammatory condition associated with elevated levels of acute-phase proteins, e.g., C-reactive protein (“CRP”). Metabolic syndrome is also associated with an increased risk of coronary artery disease, e.g., atherosclerosis and ischemic heart disease, type 2 diabetes, diseases of other end artery organs, peripheral artery disease and related conditions.

Diseases of the respiratory system are also caused by, or exacerbated by, chronic inflammation. These include, for example, asthma, bronchitis and chronic obstructive pulmonary disease (COPD). CRP is also a marker associated with systemic inflammation in COPD.

In addition, chronic inflammation is implicated in a number of diseases of the gut, such as inflammatory bowel disease or IBD. IBD includes ulcerative colitis and Crohn's disease. Skin diseases associated with inflammation include, for example, dermatitis, eczema and psoriasis. It has also been suggested that a number of diseases of the central nervous system, including Alzheimer's disease, and Parkinson's disease, are caused by, or exacerbated by, chronic inflammatory processes. Certain diseases of the musculature are also caused by, or exacerbated by, chronic inflammation, e.g., polymyositis, dermatomyositis (affects skin and muscle), inclusion body myositis (IBM) and juvenile myositis. Diseases of the joints that are caused by, or exacerbated by, chronic inflammation, such as rheumatoid arthritis, osteoarthritis, amyloidosis, ankylosing spondylitis, bursitis, psoriatic arthritis, Still's disease and others.

To date, medical treatments for chronic inflammatory conditions mainly include administering anti-inflammatory medications, such as steroidal or nonsteroidal anti-inflammatory agents. These are administered in order to reduce pain and inflammation. Sometimes analgesics, e.g., acetaminophen and/or opiates are also administered to enhance pain relief. Steroidal anti-inflammatory medications, such as betamethasone, methylprednisolone, triamcinolone, and the hundreds of analogous medications, can be administered topically, orally, by systemic injection, such as intramuscularly, intravenously, and/or by direct injection or infusion into the impacted tissue, or by inhalation for pulmonary conditions.

Non-steroidal anti-inflammatory agents (NSAIDs) can be can be administered systemically, such as orally, intramuscularly and/or intravenously, as well as topically. NSAIDs include, for example, aspirin, and its derivatives, ibuprofen, ketorolac, flurbiprofen, celecoxib, etodolac and naproxen, to name but a few such medications. Both anti-inflammatory medications and analgesics are sometimes of limited long term effectiveness, and both short and long term use of these medications raises the risk of potentially serious side effects.

Chronic inflammation has also been associated with creating a predisposition or increased risk of developing certain types of precancerous conditions (e.g., hyperplasia, metaplasia, dysplasia), and/or cancers. For example, see the review article by Schacter, et al., 2002, Oncology 16:217-26, and particularly the list of inflammatory conditions predisposing to cancer provided by Schacter et al. at Table 1. For example, gastritis caused by H. pylori is associated with a risk of gastric adenocarcinoma, chronic cholecystitis caused by certain bacteria and/or stone formation is associated with a risk of gall bladder cancer, inflammatory bowel disease is associated with a risk of colorectal carcinoma, and asbestosis or silicosis is associated with a risk of mesothelioma or lung cancer.

Exosomes are small membrane-bound particles secreted by most cell types, including stem cells, in organisms across a wide taxonomic range (Yu et al., 2014, Int J Mol Sci.7;15(3):4142-57. doi: 10.3390/ijms15034142.). Exosomes originate from internal budding of the cellular plasma membrane during endocytotic internalization, from cellular structures identified as multivesicular endosomes (MVE), that package cytoplasmic materials as membrane-bound vesicles. Exosomes have been variously reported to range in diameter from as broadly as from 30 to about 200 nm, to more particularly from about 40 to about 100 nm. Exosomes have been found to facilitate the delivery and the transfer of proteins, lipids and nucleic acids between cells. Exosomes are released from both normal and diseased cells, and are found in blood and other bodily fluids.

Exosomes have previously been shown to mediate both immunostimulatory (Zitvogel et al., US20040028692) and immunoinhibitory modulation of the immune system. Whiteside et al. 2005, British Journal of Cancer 92: 209-211). Robbins et al., US20060116321, describe the immune inhibiting properties of exosomes derived from dendritic cells.

However, there remains a longstanding need in the art for improved agents for treating chronic inflammatory conditions, as well a need for improved methods of obtaining anti-inflammatory exosomes and methods of using the same.

### SUMMARY OF THE INVENTION

Accordingly, the invention provides for anti-inflammatory exosomes and methods of making and using the same.

In a first embodiment, the invention provides a method of producing anti-inflammatory exosomes capable of inhibiting inflammation in a subject diagnosed with an inflammatory disease or disorder, the exosomes produced by a process comprising:

a) culturing animal cells in a suitable culture medium, for a time period sufficient for the culture medium to accumulate a useful concentration of anti-inflammatory exosomes, wherein the culture medium comprises an activating composition,

(b) collecting the culture medium of (a), and

(c) isolating anti-inflammatory exosomes from the culture medium collected in (b);

wherein the activating composition comprises at least one of:

(i) fluid extracted from inflamed tissue or other anatomical structure of a subject diagnosed with the inflammatory disease or disorder,

(ii) blood, plasma or serum from a subject diagnosed with the inflammatory disease or disorder,

(iii) the fluid of (i) or (ii) further comprising at least one cytokine at a concentration sufficient to enhance the effectiveness of the activating composition comprising (i) or (ii),

(iv) one or more cytokines at a concentration sufficient to induce the animal cells to secrete anti-inflammatory exosomes capable of inhibiting inflammation in a subject, and the activating composition excludes the fluid of (i) and/or the blood, plasma or serum of (ii).

Preferably, the at least one cytokine of (iii) or (iv) is interferon-gamma (IFNγ), interleukin- 1α (IL-1α), interleukin-1-β (1IL-1β), interleukin-6 (IL-6), interleukin 12b (IL-12b), tumor necrosis factor-α (TNFα) and/or combinations thereof. The cytokine is present in a concentration ranging from about 10 ng/m1 to about 50 ng/ml, and the time period for the culturing of step (a) ranges from about 24 to about 72 hours. Alternatively, the time period for the culturing of step (a) ranges from about 3 to about 6 days.

Step (c) of isolating the inflammatory exosomes is conducted by any art known method, including, without limitation, polymer precipitation, immunological separation, magnetic immunocapture, ultracentrifugation, density gradient centrifugation, size exclusion chromatography, and/or ultrafiltration.

Preferably, the animal cells employed in the inventive method are mammalian cells, and more preferably human cells, although other animal cells types, including avian cells, may optionally be employed. The animal cells are derived from any tissue or cell type that is suitable for the purpose, including, without limitation, cells derived from umbilical cord blood, placenta, non-fetal cells found in amniotic fluid, adipose tissue, bone marrow, peripheral blood, hair follicles, the gastrointestinal organs, nervous system, i.e., central and/or peripheral nervous system, circulatory system, respiratory system, the immune system, and/r secretory organs.

In certain particular aspects of the invention the animal cells are not stem cells or cancer cells.

The subject can be any animal in need thereof that will favorably respond to the administration of the inventive exosomes, including, for example, a mammal, such as a human, a canine, a feline, a porcine and an equine, or an avian.

In a second embodiment, the invention provides for anti-inflammatory exosomes produced by the above described inventive method.

In a third embodiment, the invention provides a method of treating a subject diagnosed with an inflammatory disease or disorder, by administering to the subject an effective amount of the anti-inflammatory exosomes produced by the inventive method. In certain aspects of the invention, the inflammatory disease or disorder is a tissue specific disease or disorder, such as, the inflammatory diseases resulting from metabolic X syndrome, inflammatory diseases of the gastrointestinal system, inflammatory diseases of the pulmonary system, inflammatory diseases of the skin, inflammatory diseases of the musculature, inflammatory diseases of the joints, and inflammatory diseases of the nervous system.

Alternatively, the inflammatory disease or disorder is coronary artery disease, chronic obstructive pulmonary disease (COPD), asthma, bronchitis, inflammatory bowel disease (IBD), Alzheimer's disease, Parkinson's disease, polymyositis, dermatomyositis, inclusion body myositis (IBM), juvenile myositis, rheumatoid arthritis, osteoarthritis, amyloidosis, ankylosing spondylitis, bursitis, psoriatic arthritis, Still's disease, and/or precancerous conditions.

In conducting the inventive method of treating an inflammatory disease or disorder, the anti-inflammatory exosomes are administered by any suitable art-known route, e.g., intravenous injection, intramuscular injection, intraarticular injection or infusion, subcutaneous inection, and intrathecal injection and/or infusion, as appropriate. The effective systemic amount ranges, for example, from about 1.5×10¹⁰ to about 1.5×10¹³ exosome particles per kilogram of total body weight. The effective amount for local infusion ranges, for example, from about 1.5×10¹⁰ and 1.5×10¹¹ exosome particles injected or infused into a localized tissue or anatomical space. For example, when the inflammatory disease or disorder is pulmonary, the method optionally comprises administering the anti-inflammatory exosomes as an inhaled mist or aerosol.

As noted above, the subject can be any animal in need thereof that will favorably respond to the administration of the inventive exosomes, including, for example, a mammal, such as a human, a canine, a feline, a porcine and an equine, and/or an avian.

In a fourth embodiment, the invention provides for co-treating the subject with at least one additional anti-inflammatory agent, such as an steroidal anti-inflammatory, a non-steroidal anti-inflammatory, an anti-inflammatory anti-TNF alpha antibody and combinations thereof.

In a fifth embodiment of the invention, the invention provides for a pharmaceutical composition comprising the anti-inflammatory exosomes, and a physiologically acceptable carrier suitable for systemic injection, local infusion and/or inhalation therapy.

### DETAILED DESCRIPTION OF THE INVENTION

The present invention provides anti-inflammatory exosomes and methods of obtaining and using anti-inflammatory exosomes to inhibit or downregulate the immune system inflammatory response in a subject. The subject is broadly any animal, including a mammal and/or avian, and in particular embodiments the animal is a human or veterinary subject in need of treatment thereof.

The invention also provides immunotherapy employing the inventive antiinflammatory exosomes for treating or preventing cancer, or a precancerous condition, in a subject by downregulating or inhibiting inflammatory processes that drive certain cancers or precancerous conditions.

In order to appreciate the present invention, the following terms are defined. Unless otherwise indicated, the terms listed below will be used and are intended to be defined as stated, unless otherwise indicated. Definitions for other terms can occur throughout the specification. It is intended that all singular terms also encompass the plural, active tense and past tense forms of a term, unless otherwise indicated.

Anti-inflammatory exosomes according to the invention are exosomes that when administered to a subject having an inflammatory disease or disorder, will inhibit or downregulate the inflammatory process. The anti-inflammatory exosomes are produced from cells or tissues that have been activated to enhance the immunosuppressive activity of exosomes produced or secreted by those cells or tissues. The process broadly includes contacting, e.g., culturing, suitable cells or tissues with an appropriate activating composition. Once contacted with the activating composition, the treated cells or tissues release anti-inflammatory exosomes that, when collected, purified and administered to a subject diagnosed with an inflammatory disease or disorder, will inhibit or downregulate inflammation in the treated subject.

The term, cells or tissues, as used herein, is intended to include broadly, any normal cells or normal tissues derived from or extracted from, the tissues, blood or body fluids of an animal, such as a mammal. In certain alternative embodiments, this definition excludes tumor or cancer calls or tissues.

The phrase “consisting essentially of” means that the composition or method may include additional ingredients and/or steps, but only if the additional ingredients and/or steps do not materially alter the basic and novel characteristics of the claimed composition or method, i.e., the additional ingredient and/or step(s) would serve no purpose material to the claimed composition or method.

In certain embodiments of the invention, the cells or tissues are derived, without limitation, from umbilical cord, placenta, non-fetal cells found in amniotic fluid, adipose tissue, bone marrow, peripheral blood, hair follicles, the gastrointestinal organs, nervous system, i.e., central and/or peripheral nervous system, circulatory system, respiratory system, the immune system, and secretory organs such as the mammary glands.

Cells or tissues derived from gastrointestinal organs include, without limitation, cells or tissues derived from the mucosal surface, myenteric plexus, smooth muscle and/or glandular tissues of the esophagus, stomach, small intestine, large intestine, liver, pancreas, gall bladder, salivary glands, and other gastrointestinal storage and/or secretory organs.

Cells or tissues derived from nervous system tissue, include those derived from the central nervous system, including the brain, retinas, and spinal cord. Cells or tissues derived from nervous system tissue also include those derived from the peripheral nervous system.

Cells or tissues derived from the circulatory system include those derived from blood cells, as well as those derived from the heart, e.g., heart muscle and/or heart valves, arteries, veins, and lymphatic system.

Cells or tissues derived from the respiratory system include those derived from the lungs, bronchi, bronchioles, pharynx and nasopharynx.

Cells or tissues derived from the immune system optionally include those adult stem cells associated with the immune system that are derived from the bone marrow, spleen and peripheral tissues.

Optionally, the cells or tissues are derived from the subject to be treated.

The term “culturing” refers to the in vitro maintenance, differentiation, and/or propagation of cells in suitable media. By “enriched” is meant a composition comprising cells present in a greater percentage of total cells than is found in the tissues where they are present in an organism.

Methods for culturing cells or tissues extracted from a subject include methods known to the art. Broadly, tissue from experimental animals and/or biopsies from human or veterinary patients are employed. If the tissue is a solid tissue, the tissue is minced, cultured with collagenase to break down connective tissue, treated to neutralize the collagenase, and centrifuged and/or filtered to isolate cells characteristic of the issue.

The isolated cells are then cultured under inflammatory conditions.

The duration of the culture period can be adjusted to optimize efficiency, cell count, cell feeding and exosome accumulation. In one embodiment, the time period for culturing the cells with the activating composition ranges from about 24 to about 72 hours. In an alternative embodiment, the time period ranges from about 3 to about 6 days, and in a further alternative embodiment, for about 5 days.

Broadly, the “activating composition” according to the invention is any composition that is effective to induce a cultured animal cell to secrete anti-inflammatory exosomes. In particular embodiments, the activating composition includes one or more of the following:

(i) fluid extracted from inflamed tissue, or other anatomical structure, of a mammal diagnosed with an inflammatory disease or disorder,

(ii) blood, plasma or serum from a mammal diagnosed with an inflammatory disease or disorder,

(iii) at least one cytokine at a concentration sufficient to enhance the effectiveness of the activating composition comprising (i) or (ii), or

(iv) at least one cytokine at a concentration sufficient to induce the cultured cells to secrete anti-inflammatory exosomes capable of inhibiting inflammation in a mammal, and the activating composition excludes the fluid of (i) and/or the blood, plasma or serum of (ii).

Preferably, the at least one cytokine of (iii) or (iv) is selected from the group consisting of interferon-gamma (IFNγ), interleukin-1α (IL-1α), interleukin-1-β (1 IL-1β), interleukin-6 (IL-6), interleukin 12b (IL-12b), tumor necrosis factor-α (TNFα) and combinations thereof, and is present in a concentration ranging from about 10 ng/m1 to about 50 ng/ml. Optionally, the cytokine of (iii) and/or (iv) is from an exogenous source.

An “exogenous cytokine” is a cytokine that is added from a source outside the culture medium and that is added to the culture medium to a level or concentration above that which is found in the fluid, blood, plasma or serum obtained from the subject.

The embodiment of (iv) provides for a synthetic activating composition that includes one or more cytokines, and optionally other agents, that induce the cultured cells to secrete anti-inflammatory exosomes while excluding the fluid, blood, serum and/or plasma obtained from a subject having an inflammatory condition. Optionally, the synthetic activating composition is prepared in the form of liposomes designed to mimic the properties and composition of exosomes, preferably ranging in size from about 40 nm to about 100 nm, with a density between 1.15 g/ml (Lane et al., 2015, Scientific Reports 5, Article number: 7639 doi:10.1038/srep07639).

The synthetic activating composition includes, without limitation, cytokines, such IFNγ, TNFα, IL1α and IL1β, in concentrations ranging from about 10 ng/ml to about 50 ng/ml.

In other embodiments of the invention, the cells may be genetically engineered to express a gene or genes encoding one or more heterologous activating agents. Such genes would encode cytokines, including IFNγ, TNFα, IL1α and/or IL1β. Alternatively, the cultured cells may be grown on a substrate of supporting cells, such as fibroblasts, engineered to express the activating agents listed above.

Culture media for culturing mammalian cell lines, in vitro are known to those skilled in the art and commonly used. For example, a suitable culture medium is Eagle's minimal essential medium with 10% Fetal Bovine Serum, 10 mL/L Pen/Strep Solution, 2 mM Ala-Gln solution, 10 ng/ml Epidermal Growth Factor, 10 μg/ml Insulin solution, 100 μM 2-fosfo-L-ascorbic acid trisodium salt, and 0.01 μM dexamethasone.

Cells are cultured, for example, by inoculating culture medium, with from about 30,000 to about 50,000 cells per ml.

After incubating for from about 2 to about 4 days at 37° C., the inoculated culture medium is collected and the exosomes purified and isolated from the culture medium. This can be accomplished by any suitable art-known method. For example, see Robbins et al., US20060116321 or Lane et al., Id., Brownlee, et al., 2014, J Immunol Methods, 407: 120-126. doi: 10.1016/j.jim.2014.04.003. These methods include, for example, the original method of separating exosomes by differential ultracentrifugation, and newer methods, such as polymer precipitation (ExoQuick™ from SBI, Palo Alto, Calif.), immunoaffinity capture (Greening et al. 2015, Methods in Molecular Biology, Impact Factor: 1.29), immune magnetic capture (Exo-FLOW™, SBI), the Invitrogen Total Exosome Isolation Kit (Life Technologies, USA) and the ExoSpin Exosome Purification Kit (Cell Guidance Systems, USA).

Immuno-affinity purification is a method to selectively capture specific exosomes based upon surface markers. This approach employs magnetic beads covalently coated with streptavidin, which can be coupled in high affinity fashion with biotinylated capture antibody. Captured exosomes are eluted and are intact and bioactive.

Purified exosomes are quantified by determining the protein content and the activity of acetyl-CoA acetetylcholinesterase, and are analyzed for size distribution and concentration by nanoparticle tracking analysis. Isolated exosomes are validated for exosomal marker expression by flow cytometry and Western blot.

The invention also provides methods of treating subjects, including subjects, diagnosed with a disease or disorder caused by, or exacerbated by, an inflammatory disorder and/or requiring modulation of the immune system. When the subject is a human subject, such diseases or disorders are contemplated to include, without limitation, arthritis, allergy, asthma, or an autoimmune disease such as, rheumatoid arthritis, osteoarthritis, juvenile rheumatoid arthritis, systemic lupus erythematosis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo. Other conditions which may desirably be treated include diseases such as muscular dystrophy, and conditions in which inflammation can interfere with proper healing, such as an accidental or iatrogenic wound in soft tissue, ligament, or bone, or tissue damaged by a non-immune event, for example, heart muscle following myocardial infarction.

The diseases or disorders contemplated to be treated according to the invention include both systemic and tissue specific diseases or disorders. Systemic diseases include, for example, the various manifestations of metabolic syndrome, such as coronary artery disease, e.g., atherosclerosis and ischemic heart disease, type 2 diabetes, diseases of other end artery organs, peripheral artery disease and related conditions.

Tissue specific diseases include inflammatory diseases confined to a particular organ or tissue type, as follows.

Diseases or disorders of the respiratory system to be treated include, for example, asthma, bronchitis and chronic obstructive pulmonary disease (COPD). Diseases or disorders of the gastrointestinal system to be treated include, for example, inflammatory bowel disease or IBD, such as ulcerative colitis and Crohn's disease. Skin diseases to be treated include, for example, dermatitis, eczema and psoriasis. Diseases or disorders of the central nervous system to be treated include, for example, Alzheimer's disease, Parkinson's disease and optionally migraine conditions. Diseases or disorders of the musculature to be treated include, for example, polymyositis, dermatomyositis, inclusion body myositis (IBM) and juvenile myositis. Diseases or disorders of the joints to be treated include, for example, rheumatoid arthritis, osteoarthritis, amyloidosis, ankylosing spondylitis, bursitis, psoriatic arthritis, Still's disease and others.

In certain embodiments, the inflammatory diseases or disorders are those predisposing to a higher risk of cancer, such as, for example, the various inflammatory gastrointestinal diseases, such as inflammatory bowel disease and Barrett's esophagous; chronic bacterial infections, e.g., infection with H. pylori, chronic asbestosis, silicosis and other tissue inflammations caused by inhaling or ingesting non-biodegradable dusts, infections with parasites such as Schistosomiasis, infections with viruses, such as the Epstein-Barr virus, human papilloma virus, hepatitis B virus, and human herpes virus-8, chronic inflammation induced by exposure to tobacco products and so forth.

In another embodiment, the inflammatory disease or disorder to be treated is osteoarthritis, and the activating composition includes, without limitation, synovial fluid from one or more inflamed joints of the osteoarthritic mammal. The mammalian subject can be a human subject, or a veterinary subject, such as, for example, and without limitation, domesticated animals, animals typically kept as pets or work animals, and or exotic animals, e.g., zoo animals, for which it is desired to treat an inflammatory disorder. For example, it is contemplated that the inventive methods be applied, without limitation to subjects that include humans and veterinary subjects. Veterinary subjects include mammals and avians. In addition to humans, mammalian subjects include, simply by way of example, non-human primates, bovine (e.g., cattle or dairy cows), porcine (e.g., hogs or pigs), ovine (e.g., goats or sheep), equine (e.g., horses), canine (e.g., dogs), feline (e.g., house cats), camels, deer, antelopes, rabbits, guinea pigs and rodents (e.g., squirrels, rats, mice, gerbils, and hamsters), cetaceans (whales, dolphins, porpoise), pinnipeds (seals, walrus). Avian subjects include, simply by way of example, Anatidae (e.g., swans, ducks and geese), Columbidae (e.g., doves and pigeons), Phasianidae (e.g., partridges, grouse and turkeys) Thesienidae (e.g., domestic chickens), Psittacines (e.g., parakeets, macaws, and parrots), game birds, and ratites, (e.g., ostriches).

The invention also provides purified anti-inflammatory exosomes prepared by the above described methods.

Without meaning to be bound by any theory or hypothesis as to how the invention operates, anti-inflammatory exosomes produced by cells or tissues cultured in the presence of an activating composition, which includes factors secreted from inflammatory tissue, induce macrophages present in inflamed tissues to change from an M1 pro-inflammatory phenotype to the M2 macrophage immunosuppressive phenotype.

M1 macrophages are pro-inflammatory cells with potent anti-microbial activity that promote T helper cell responses. M1 macrophages have also been implicated in many inflammatory disease, such as osteoarthritis. M2 macrophages are immunosuppressive cells that can support T helper cell 2 (Th cell 2) associated effector functions. M2 macrophages, produce anti-inflammatory cytokines (Rőszer T, 2015, Mediators Inflamm. 2015:816460. doi: 10.1155/2015/816460), and are thought to play a major role in the resolution of inflammation, tissue remodeling and in wound repair.

The anti-inflammatory exosomes are administered by any clinically appropriate route to deliver the exosomes to the inflamed tissue or organ, or may be delivered systemically when clinically appropriate. By way of example, the anti-inflammatory exosomes are administered by a route such as, intravenously, intramuscularly, intraarticularly, subcutaneously and/or intrathecally and/or by direct injection, infusion or instillation, intranasally or by inhalation, into an inflamed tissue or organ, as well as topically to the skin.

An “effective amount” is an amount sufficient to effect beneficial or desired results, such as a downregulated inflammatory response, treatment, prevention or amelioration of a medical condition (disease, infection, etc.). An effective amount can be administered in one or more administrations, applications or dosages. The effective amount, i.e., a suitable dosage, will vary depending on body weight, age, health, disease or condition to be treated and route of administration. The dose of exosomes administered to a subject is in an amount effective to achieve the desired beneficial therapeutic response in the subject over time.

The artisan will be readily able to determine the amount of exosomes to be administered by titrating the dose and duration of administration to reach an optimal clinical response, such as a reduction in the inflammatory process of the disease or disorder that is being treated.

In particular, the anti-inflammatory exosomes are administered systemically, in an amount ranging from about 1.5×10¹⁰ to about 1.5×10¹³ exosome particles per kilogram of total body weight. Aternatively, the anti-inflammatory exosomes are administered in an amount ranging from about 1.5×10¹⁰ and 1.5×10¹¹ exosome particles injected or infused into a localized tissue or anatomical space.

The number of exosomes in a preparation can be determined by any art-known method. In a non-limiting example, exosome particle numbers can be determined by direct counting using a NanoSight instrument, such as a NanoSight® NS300, NanoSight NS500® or NanoSight® LM10 (Malvern Instruments, Ltd, Worcestershire, UK). Alternatively, the number of exosomes can be estimated by measuring the activity of Acetyl-CoA acetetylcholinesterase, an enzyme present within exosomes, and then estimating the exosome count by reference to a pre-prepared standard curve of exosome counts verses Acetyl Co-A levels.

The treatment is repeated as needed until a positive anti-inflammatory result is obtained. Optionally, the treatment is repeated at a daily, weekly or monthly interval, as needed, in order to maintain suppression of the inflammatory process.

In a further embodiment, the invention contemplates co-treating a subject in need thereof, with at least one additional anti-inflammatory agent, the at least one additional anti-inflammatory agent including, for example, a steroidal anti-inflammatory, a non-steroidal anti-inflammatory, an anti-TNF alpha antibody and combinations thereof.

Steroidal anti-inflammatory medications include, without limitation, cortisone, triamcinolone, dexamethasone, hydrocortisone, prednisone, methylprednisolone, prednisolone hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Steroidal anti-inflammatory medications formulated for inhalation therapy include, without limitation, beclomethasone, budesonide, ciclesonide, flunisolide, fluticasone, and triamcinolone.

Non-steroidal anti-inflammatory drugs (NSAIDs) represent a large group of therapeutic agents with analgesic, anti-inflammatory, and anti-pyretic properties. NSAIDs typically reduce inflammation by blocking the cyclooxygenase 1 and/or cyclooxygenase 2 (COX 1 and COX 2) enzymes. NSAIDs that selectively inhibit COX 2 enzymes are more sparing of the gastric mucosa, where COX1 is predominant. Representative NSAIDs include, without limitation.

Non-steroidal anti-inflammatory drugs (NSAIDs) represent a large group of therapeutic agents with analgesic, anti-inflammatory, and anti-pyretic properties. NSAIDs typically reduce inflammation by blocking the cyclooxygenase 1 and/or cyclooxygenase 2 (COX 1 and COX 2) enzymes. NSAIDs that selectively inhibit COX 2 enzymes are more sparing of the gastric mucosa, where COX1 is predominant. Representative NSAIDs include, without limitation, aceclofenac, acemetacin, actarit, alcofenac, alminoprofen, amfenac, aloxipirin, aspirin, azapropazone, benzydamine (prostaglandin synthase inhibitor), butibufen, celecoxib, chlorthenoxacin, choline salicylate, dexketoprofen, diclofenac, diflunisal, emorfazone, epirizole; etodolac, etoricoxib, feclobuzone, felbinac, fenbufen, fenclofenac, fenoprofen, flurbiprofen, glafenine, hydroxylethyl salicylate, ibuprofen, indomethacin, indoprofen, ketoprofen, ketorolac, loxoprofen, lumiracoxib, mefenamic acid, meloxicam, metamizole, mefenamic acid, metiazinic acid, mofebutazone, mofezolac, nabumetone, naproxen, nifenazone, niflumic acid, oxaprozin, piroxicam, pranoprofen, propyphenazone, proquazone, protizinic acid, rofecoxib, salicylamide, salsalate, sulindac, suprofen, tiaramide, tinoridine, tolfenamic acid, tolmetin, and valdecoxib.

Antibody based anti-inflammatory medications include, without limitation, infliximab, etanercept, alemtuzumab, adalimumab, omalizumab, efalizumab, alefacept, natalizumab, abatacept, certolizumab pegol, golimumab, canakinumab, tocilizumab, ustekinumab (MAbs. 2010 May-Jun; 2(3): 233-255), Vedolizumab, talizumab, abrilumab, inclacumab, anifrolumab, anrukinzumab, benralizumab, brodalumab, clazakizumab, clenoliximab, eldelumab, etrolizumab, gomiliximab, mavrilimumab, oxelumab, pateclizumab, perakizumab, quilizumab, rontalizumab, sirukumab, tezepelumab, Tildrakizumab, and zanolimumab.

### EXAMPLES

The following examples are provided in order to illustrate the present invention, without intending to limit the scope of the present invention.

### Example 1

### Isolation of Cells From an Inflamed Tissue

A biopsy sample of tissue exhibiting characteristic metaplasia is obtained from the esophagus of a patient diagnosed with Barrett's esophagus. Cells are isolated essentially as described by Secunda R, 2015, Cytotechnology. 67(5):793-807. doi: 10.1007/s10616-014-9718-z. The biopsy sample is minced and digested with 0.075% collagenase type I for 30 min at 37° C., and then the collagenase type I activity is neutralized by adding α-MEM containing 20% heat inactivated fetal bovine serum.

After neutralization of the enzyme, the sample is centrifuged at 600× g for 10 minutes and filtered through 70 μm cell strainer. Freshly isolated cells (1.5×10⁶) are plated onto 100 mm dishes in Eagle's minimal essential medium with 10% Fetal Bovine Serum, 10 mL/L Pen/Strep Solution, 2 mM Ala-Gln solution, 10 ng/ml Epidermal Growth Factor, 10 μg/ml Insulin solution, 100 μM 2-fosfo-L-ascorbic acid trisodium salt, and 0.01 μM Dexamethasone.

The culure medium is replaced with fresh medium every 4 days. Once the cells reach 70 to 80% confluence, they are detached by TrypLE Express and replated at a density of 1×10³/cm² to 2×10³/cm².

### Example 2

### Culture of Isolated Cells Under Inflammatory Conditions with Activating Composition

Cells are cultured, for example, by inoculating culture medium, with from about 30,000 to about 50,000 cells per ml.

The cells obtained in Example 1 are incubated in suitable culture medium, under inflammatory conditions. Inflammatory conditions are provided by adding to the culture medium one or more pro-inflammation cytokines, and/or fluid extracted from inflamed tissues to the culture medium at 37° C. After a time in culture sufficient for the accumulation of useful levels of anti-inflammatory exosomes, e.g., from about 2 to about 4 days, the cultured cells are separated from the culture medium, and the culture medium is collected.

### EXAMPLE 3

### Isolating Anti-Inflammatory Exosomes

Exosomes are purified and isolated from the culture medium collected in Example 2 by polymer precipitation (ExoQuick™ from SBI, Palo Alto, Calif.), immunoaffinity capture (Greening et al. 2015, Methods in Molecular Biology, Impact Factor: 1.29).

The exosomes isolated by polymer precipitation are further purified by being bound to magnetic beads (Exo-FLOW™, SBI). Magnetic beads [9.1 μm, 1.6×107 beads/ml] are coupled with anti-CD9 or anti-CD63 or anti-CD81 biotinylated antibody for 2 h on ice, and then incubated with exosomes on a rotating rack at 4° C. overnight for capture. The beads are coated with the three different antibodies separately and then mixed for the capture of exosomes.

### Example 4

### Validating Isolated Exosomes

The exosomes purified by Example 4 are quantified by determining the protein content and the activity of acetyl-CoA acetetylcholinesterase, and are analyzed for size distribution and concentration by nanoparticle tracking analysis. Isolated exosomes are validated for exosomal marker expression by flow cytometry and Western blot.

Exosomes are validated by Western blot using the specific exosomal marker TSG101 and by flow cytometry using Exo-FITC™ staining. This staining takes advantage of the finding that most exosome surface proteins have modifications, such as, glycosylations, carbohydrate additions, etc. that are bound by the protein component of SBI's protein-fluorescein isothiocyanate (FITC) conjugate, commercially available as Exo-FITC™ (SBI). The data indicate that about 90% of the exosomes bound to the beads are positive for the staining. The SF-derived exosomes are analyzed for size distribution and concentration by Nanosight™.

### Example 5

### Confirming Ant-Inflammatory Activity of Isolated Anti-Inflammatory Exosomes

The M1-stimulatory properties of exosomes produced and purified by Examples 1-4 is evaluated by cytokine gene expression analysis. M1 macrophages are incubated with the purified exosomes for 6 h and cytokine coding mRNA expression is evaluated by RT-PCR. A significant upregulation in gene expression of IL-1β is observed, together with a down regulation of the expression of IL12b.

The overall results confirm that synovial fluid-derived exosomes are able to stimulate M1 macrophages.

### INCORPORATION BY REFERENCE

All publications cited herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

We claim:

1. A method of producing anti-inflammatory exosomes capable of inhibiting inflammation in a subject diagnosed with an inflammatory disease or disorder, the exosomes produced by a process comprising:
a) culturing animal cells or tissues in a suitable culture medium, for a time period sufficient for the culture medium to accumulate a useful concentration of anti-inflammatory exosomes, wherein the culture medium comprises an activating composition,
(b) collecting the culture medium of (a), and
(c) isolating anti-inflammatory exosomes from the culture medium collected in (b);
wherein the activating composition comprises at least one of:
(i) fluid extracted from inflamed tissue or other anatomical structure of a mammal diagnosed with the inflammatory disease or disorder,
(ii) blood, plasma or serum from a subject diagnosed with the inflammatory disease or disorder,
(iii) the fluid of (i) or (ii) further comprising at least one cytokine at a concentration sufficient to enhance the effectiveness of the activating composition comprising (i) or (ii),
(iv) one or more cytokines at a concentration sufficient to induce the animal cells to secrete anti-inflammatory exosomes capable of inhibiting inflammation in a subject, and the activating composition excludes the fluid of (i) and/or the blood, plasma or serum of (ii).

2. The method of claim 1, wherein the at least one cytokine of (iii) or (iv) is selected from the group consisting of interferon-gamma (IFNγ), interleukin-1α(IL-1α), interleukin-1-β (1IL-1β), interleukin-6 (IL-6), interleukin 12b (IL-12b), tumor necrosis factor-α(TNFα) and combinations thereof.

3. The method of claim 2, wherein the at least one cytokine is present in a concentration ranging from about 10 ng/ml to about 50 ng/ml.

4. The method of claim 1, wherein the time period for the culturing of step (a) ranges from about 24 to about 72 hours.

5. The method of claim 1, wherein the time period for the culturing of step (a) ranges from about 3 to about 6 days.

6. The method of claim 1, wherein the anti-inflammatory exosomes are able to transform M1 macrophages to M2 macrophages.

7. The method of claim 1, wherein the anti-inflammatory exosomes are isolated from the culture medium of (a) by a method selected from the group consisting of polymer precipitation, immunological separation, magnetic immunocapture and combinations thereof.

8. The method of claim 1, wherein the anti-inflammatory exosomes are isolated from the culture medium of (a) by a method selected from the group consisting of ultracentrifugation, density gradient centrifugation, size exclusion chromatography, and ultrafiltration.

9. The method of claim 1, where the inflammatory disease or disorder is a tissue specific disease or disorder.

10. The method of claim 1, wherein the inflammatory disease or disorder is selected from the group consisting of the inflammatory diseases resulting from metabolic X syndrome, inflammatory diseases of the gastrointestinal system, inflammatory diseases of the pulmonary system, inflammatory diseases of the skin, inflammatory diseases of the musculature, inflammatory diseases of the joints, and inflammatory diseases of the nervous system.

11. The method of claim 1, wherein the inflammatory disease or disorder is selected from the group consisting of coronary artery disease, chronic obstructive pulmonary disease (COPD), asthma, bronchitis, inflammatory bowel disease (IBD), Alzheimer's disease, Parkinson's disease, polymyositis, dermatomyositis, inclusion body myositis (IBM), juvenile myositis, rheumatoid arthritis, osteoarthritis, amyloidosis, ankylosing spondylitis, bursitis, psoriatic arthritis, Still's disease, and precancerous conditions.

12. The method of claim 1, wherein the animal cells are mammalian cells.

13. The method of claim 1, wherein the animal cells are human cells.

14. The method of claim 1, wherein the animal cells are cells derived from tissues selected from the group consisting of umbilical cord blood, placenta, non-fetal cells found in amniotic fluid, adipose tissue, bone marrow, peripheral blood, hair follicles, the gastrointestinal organs, nervous system, i.e., central and/or peripheral nervous system, circulatory system, respiratory system, the immune system, and secretory organs.

15. The method of claim 14, wherein the animal cells are not stem cells and are not cancer cells.

16. The method of claim 1, wherein the subject is a mammal is selected from the group consisting of a human, a canine, a feline, a porcine and an equine.

17. Anti-inflammatory exosomes produced by the method of claim 1.

18. A method of treating a subject diagnosed with an inflammatory disease or disorder, comprising administering to the subject an effective amount of the anti-inflammatory exosomes of claim 17.

19. The method of claim 18, wherein the inflammatory disease or disorder is a tissue specific disease or disorder.

20. The method of claim 18, wherein the inflammatory disease or disorder is selected from the group consisting of the inflammatory diseases resulting from metabolic syndrome, inflammatory diseases of the gastrointestinal system, inflammatory diseases of the pulmonary system, inflammatory diseases of the skin, inflammatory diseases of the musculature, inflammatory diseases of the joints, and inflammatory diseases of the nervous system.

21. The method of claim 18, wherein the inflammatory disease or disorder is selected from the group consisting of coronary artery disease, chronic obstructive pulmonary disease (COPD), asthma, bronchitis, inflammatory bowel disease (IBD), Alzheimer's disease, Parkinson's disease, polymyositis, dermatomyositis, inclusion body myositis (IBM), juvenile myositis, rheumatoid arthritis, osteoarthritis, amyloidosis, ankylosing spondylitis, bursitis, psoriatic arthritis, and Still's disease.

22. The method of claim 18, wherein the anti-inflammatory exosomes are administered by a route selected from the group consisting of, intravenous injection, intramuscular injection, intraarticular injection or infusion, subcutaneous inection, and intrathecal injection or infusion.

23. The method of claim 18, wherein the inflammatory disease or disorder is pulmonary, and the method comprises administering the anti-inflammatory exosomes as an inhaled mist or aerosol.

24. The method of claim 18, wherein the subject is a mammal selected from the group consisting of a human, a canine, a porcine, a feline and an equine.

25. The method of claim 18, wherein the anti-inflammatory exosomes are administered systemically, in an amount ranging from about 1.5×10¹⁰ to about 1.5×10¹³ exosome particles per kilogram of total body weight.

26. The method of claim 18, wherein the anti-inflammatory exosomes are administered in an amount ranging from about 1.5×10¹⁰ and 1.5×10¹¹ exosome particles injected or infused into a localized tissue or anatomical space.

27. The method of claim 18, further comprising co-treating the subject with at least one additional anti-inflammatory agent, the at least one additional anti-inflammatory agent selected from the group consisting of a steroidal anti-inflammatory, a non-steroidal anti-inflammatory, an anti-inflammatory anti-TNF alpha antibody and combinations thereof.

28. A pharmaceutical composition comprising the anti-inflammatory exosomes of claim 17, and a physiologically acceptable carrier.
